(19) United States
(12) Patent Application Publication
(10) Pub. No.: US 2024/0285827 A1
(43) Pub. Date: Aug. 29, 2024

(54) OXYGEN-ENRICHED SILICONE OIL AND PERFLUOROCARBON AS VITREORETINAL TAMPONADE

(71) Applicant: OPTICGON, LLC., Flushing, NY (US)

(72) Inventor: Ian H. CHAN, Flushing, NY (US)

(21) Appl. No.: 18/587,837

(22) Filed: Feb. 26, 2024

Related U.S. Application Data
(60) Provisional application No. 63/448,616, filed on Feb. 27, 2023.

(51) Int. Cl. A61L 26/00; A61F 9/00; A61K 33/00; A61K 47/06; A61K 47/44; A61P 27/02

(52) U.S. Cl. CPC A61L 26/0004; A61F 9/0017; A61K 33/00; A61K 47/06; A61K 47/44; A61L 26/0066; A61L 26/0095; A61P 27/02; A61L 2300/10

(57) ABSTRACT

Described herein are oxygen-enriched tamponade compositions comprising silicone oil or a perfluorocarbon and methods of their use in the repair of retinal detachment. Post-operative repair for retinal detachment can include enhanced wound healing as the result of the administration of the compositions described herein. The enhanced wound healing can include rescue of ischemic retinal tissue, surgery induced hypoxia, and reduction of unwanted scar tissue development and prevention of re-detachment of the retina after surgical repair.

### RELATED APPLICATIONS

This disclosure claims priority to U.S. Provisional Patent Application No. 63/448,616, filed Feb. 27, 2023, the contents of which are herein incorporated by reference.

### FIELD

The present invention relates to vitreoretinal tamponades and particularly to silicone and perfluorocarbon tamponades that are configured to be injected as a vitreous substitute in the retina of a patient's eye. The present invention creates a vitreous substitute with enriched-oxygen environment to reduce tissue hypoxia and enhance wound healing of the retina in the post-operative period.

### BACKGROUND

The following includes information that may be useful in understanding the present invention. It is not an admission that any of the information, publications or documents specifically or implicitly referenced herein is prior art, or essential, to the presently described or claimed inventions. All publications and patents mentioned herein are hereby incorporated by reference in their entirety.

Retinal detachment involves the physical separation of the neuroretina from the supporting retinal pigment epithelium (RPE) monolayer. Rhegmatogenous retinal detachment can often occur, which is the detachment, and subsequent tearing, of the retina, whereby aqueous from around the vitreous humour can accumulate between the two layers leading to further separation from the RPE. Retinal detachment may occur without the formation of a tear either by the buildup of fluid under the retina from blood vessels, so called exudative retinal detachment, triggered by conditions such as severe macular degeneration, very high blood pressure or certain cancers such as choroidal melanoma. Proliferative vitreoretinpathy (PVR) is a known complication of rhegmatogenous retinal detachment and recurring retinal detachment because the formation of a tear in the retina results in the production of an inflammatory microenvironment.

Poor wound healing and scar tissue formation following surgical repair often leads to re-detachment of the retina after surgical repair. Re-detachment is more common in cases where retinal ischemia is present, such as in patients with severe diabetes and tractional retinal detachment (TRD), and cases with significant intraoperative bleeding and cases with proliferative vitreoretinpathy (PVR). Both PVR and TRD cases involve a highly ischemic retina and both present a profound development of scar tissue, pre-and sub-retinal retinal fibrotic membranes which both destroy the retinal tissue itself, as well as the creation of traction on the retina leading to its detachment.

Therapeutic intervention for retinal detachment includes the use of ocular tamponades. A tamponade agent is a material that will plug or compress an area or cavity. Ocular tamponades are vitreous substitutes that are used to reposition the retina of an eye in instances where a reattachment is not completely achievable by natural healing or by laser coagulation. Ocular tamponades include medical gas (i.e., C3F8 and SF6), perfluorocarbon-liquids (perfluorocarbons) or silicone oil (polydimethysiloxane). Silicone oil is the preferred tamponade for high-risk cases with retinal ischemia and hypoxia. Gas tamponades (Pneumatic retinopexy) involve the injection of a gas bubble (air or medical gas) into the vitreous cavity and careful positioning of the patients' head to align the bubble with the detached area, which then generates a light pressure which is able to hold the tear against the RPE. The process takes up to three weeks, during which the gas is slowly absorbed into the body. Gas tamponades also result in significant restrictions for the patient including postural and lifestyle limitations. The use of pure gas tamponades also presents hazards to the patient including cataract formation risk and a steep ride in interocular pressure (IOP) (Selim et al., ARC Journal of Ophthalmology, Volume-4 Issue-1, 2019, Page No: 6-8).

### SUMMARY

The inventions described and claimed herein have many attributes and aspects including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

The present invention relates to a tamponade composition (selected from silicon oil or perfluorocarbon) enriched with oxygen. The same oxygen-enriched tamponade concept can be directly applied to perfluorocarbon as well but for the description of the present invention, silicone oil will be used as example. The silicone oil serves as a vitreoretinal tamponade and an oxygen-releasing reservoir to reduce retinal ischemia and hypoxia, and enhance wound healing in the post-operative period. Oxygen supplementation in the post-operative period can reduce the risk of serious complications such as redetachment, proliferative vitreoretinopathy and development of tractional membranes. Vitreoretinal tamponade enriched with oxygen can serve as a reservoir to supply a continuous release of oxygen in the post-operative period to the healing retinal tissue.

In some aspects, this disclosure provides for an oxygen-enriched tamponade composition comprising oxygen at a concentration higher than that of ambient concentration and a base oil selected from a silicone oil or a fluorocarbon. In some aspects, the oxygen has been exogenously added to said base oil.

The concentration of oxygen in the oxygen-enriched tamponade compositions of this disclosure are significantly higher than the oxygen concentration in virgin (no exogenous oxygen infusion) base oil. The oxygen concentration in virgin base oil is typically 0-1%, based on ambient conditions. The oxygen concentration of the oxygen-enriched tamponade compositions of this disclosure can range from at least 20-52%, or higher, compared to the levels in virgin base oil.

In some aspects, this disclosure provides for a method of preparing an oxygen-enriched tamponade composition comprising adding exogenous oxygen to a base oil. The base oil can be selected from a silicone oil, a fluorocarbon, or mixtures thereof.

In some aspects, the exogenous addition of oxygen can be performed by directly infusing medical grade oxygen gas into the fluid tamponade. In some aspects, the direct oxygen infusion can be performed by sparging said medical grade oxygen gas into the base oil. In some aspects, the direct oxygen infusion can be performed by inline static mixing of oxygen gas into the base oil. In some aspects, the direct oxygen infusion can be performed by dynamic mixing injected oxygen gas with a rotor to stir the base oil and injecting oxygen gas simultaneously.

In some aspects, the direct oxygen infusion can be performed by injection of microbubbles comprising oxygen into the base oil. The size of the microbubbles can range from 1 to 500 microns. In some aspects, the size of the microbubbles is less than 50 microns. In some aspects, the microbubbles can be formed by injecting oxygen gas into a micro-cannula configured to be embedded within a surrounding cannula which comprises the base oil followed by expelling the mixture from the cannulas to yield the oxygen-enriched tamponade composition. The micro-cannula and surrounding cannula can each comprise a separate lumen, and the lumen can be configured to merge at an expelling point. Mixing of the microbubbles can occur when the micro-cannula and surrounding cannula lumen merge together.

In some aspects, the direct oxygen infusion can be performed by the creation of cavitation or bubbles by ultrasonicating the base oil while simultaneously injecting oxygen gas into said base oil.

In some aspects, the methods of preparing oxygen-enriched tamponade compositions of this disclosure can be performed using exogenous oxygen pressure added at a range from 20-3000 psi.

In some aspects, the methods of preparing oxygen-enriched tamponade compositions of this disclosure can be performed at a temperature ranging from −10 to +100 degrees Celsius. In some aspects, the silicone oil can be cooled to about −8 degrees Celsius and then oxygen can be introduced into the silicone oil by sparging to enhance the levels of dissolved oxygen in the silicone oil.

In some aspects, this disclosure provides for a method of treating a retinal injury in a subject comprising the steps of: creating an incision into the posterior chamber of an eye of a subject having a retinal injury; and administering to said posterior chamber of the eye an oxygen-enriched tamponade as described herein. In some aspects, the oxygen-enriched fluid tamponade can be stored in air-tight vessel to maintain the oxygen content prior to its use as an ocular tamponade. In some aspects, the oxygen in the oxygen-enriched tamponade can be continuously released for up to 7 days after administration. In some aspects, the oxygen in the oxygen-enriched tamponade can be continuously released for up to 2 days after administration. In some aspects, 80% of the oxygen in the oxygen-enriched tamponade is continuously released for up to 2 days after administration.

These and other objectives and advantages of the present invention, some of which are specifically described and others that are not, will become apparent from the detailed description and claims that follow.

### DETAILED DESCRIPTION OF THE INVENTION

The present invention relates to a novel approach to create an oxygen-enriched vitreoretinal tamponade.

### Certain Definitions

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable, which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value, for variables which are inherently continuous.

As used herein, the use of the word “a” or “an” when used in conjunction with the term “comprising” in the claims and/or the specification may mean “one,” but it is also consistent with the meaning of “one or more,” “at least one,” and “one or more than one”. Throughout this application, the term “about” is used to indicate that a value includes the inherent variation of error for the device, the method being employed to determine the value.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open ended and do not exclude additional, un-recited elements or method steps.

As used herein, the term “subject” or “subject in need thereof” refers to humans as well as non-human animals, such as domesticated mammals including, without limitation, cats, dogs, and horses. The compositions and methods of the present invention are intended for use with any subject that may experience the benefits of the formulations and methods of the invention. The subject is typically a mammal, more typically a human. However, the invention is not limited to the treatment of humans and is applicable to veterinary uses.

As used herein, the term “therapeutically effective amount” refers to treatments at dosages effective to achieve the therapeutic result sought. The term “therapeutically effective amount” or “effective amount” means the amount of the subject compound that will elicit a desired response, for example, a biological or medical result or response of a tissue, system, animal or human that is sought, for example, by a researcher, veterinarian, medical doctor, or other clinician. That result can be alleviation of the signs, symptoms, or causes of a disease or disorder or condition, or any other desired alteration of a biological system.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “composition” refers to a product comprising one or more ingredients.

### Representative Methods of Treatment

In some embodiments, this disclosure provides for methods of treating tractional retinal detachment in the setting of diabetic retinopathy, retinal detachment in the presence of proliferative vitreoretinopathy, trauma-related retinal detachment, a highly myopic eye, in the presence of inflammation and chronic retinal detachment with delay of retinal repair surgery. In some embodiments, this disclosure provides for methods of treating proliferative retinal pigment epithelium diseases, a detached retina, a torn retina or a disease, condition or disorder associated with an abnormality in the retinal pigment epithelium or its function. In some embodiments, this disclosure provides for methods of treating proliferative vitreoretinopathy, retinal pigment epithelium cell proliferation or proliferative diabetic retinopathy. In some embodiments, the detached or torn retina may be caused by rhegmatogenous retinal detachment, exudative retinal detachment, or tractional retinal detachment.

In some embodiments, this disclosure relates to the use of an oxygen-enriched tamponade composition for use as a reservoir for oxygen delivery in the post-operative period where tissue ischemia causes poor wound healing and scar tissue formation.

Because silicone oil is a reference tamponade for the clinical treatment of high-risk cases of retinal ischemia, re-detachment and proliferative vitreoretinopathy formation, oxygen-enriched silicone oil can improve tissue hypoxia, enhance wound healing and lower the risk for re-detachment and ischemia-related complications in retinal surgery.

In some embodiments, this disclosure also provides compositions for use in a method of manufacturing a medicament for preventing or treating an eye disorder in a human or animal subject.

The methods, uses in methods and oxygen-enriched tamponade compositions disclosed herein may comprise administering to the human or animal subject a therapeutically effective amount of the composition, as defined in the present invention. In some embodiments, the methods of treating the retinal injuries of this disclosure comprise administering to the eye of a subject in need thereof the oxygen-enriched tamponade compositions of this disclosure.

### Extended Release of Oxygen

Oxygen-enriched silicone oil serves as an oxygen reservoir where there is continued release of oxygen. In some embodiments, the oxygen is released from the oxygen-enriched tamponade compositions of this disclosure over an extended period of time. In some embodiments, the release time ranges from the initial time of administration up to 1, 2, 3, 4, 5, 6, 7, 8, 9, 10, 11, 12, 13, or 14 days. In some embodiments, the release time ranges from the initial time of administration up to 7 days. In some embodiments, the oxygen release time can be from initial time of administration to up to 2 to about 10 weeks, from about 3 to about 9 weeks, from about 4 to about 8 weeks, or from about 5 to about 7 weeks.

### Representative Methods of Making Oxygen-enriched Tamponade Compositions Silicone oil

In some embodiments, the oxygen-enriched tamponades of this disclosure comprise silicone oil. The silicone oil may be polydimethylsiloxane. In some embodiments, the base oil may comprise purified polydimethylsiloxane, which can include or exclude SiO1000, SiO5000, Siluron® 2000, Siluron® 5000, Siluron® 1000 (Fluron® GmBH, Germany), or Sil-1000® or Sil-5000® (D.O.R.C. Dutch Ophthalmic Research Center (International), The Netherlands), or Oxane 1300 or Oxane 5700 (Bausch+Lomb (UK)), or Sil-Vit 1.000 or Sil-Vit 5.000 (Vitreq, The Netherlands), or mixtures thereof. In some embodiments, the base oil may comprise a high-density silicone oil that comprises polydimethylsiloxane and perfluorohexyloctane, which can include or exclude: Densiron® 68, Densiron® Xtra and Siluron® Xtra. It will be understood that other equivalent commercial products could be used that achieve a similar technical effect.

In some embodiments, the tamponade can comprise silicone oil. The silicone oil comprising tamponade can include or exclude those described in U.S. Pat. Nos. 6,703,378, 7,276,619, 5,672,355, 8,859,618, and 6,703,378; U.S. Patent Application Nos. US20,090,170811, US20,090,170811; and PCT Publication No. WO2011140101, each of which is incorporated by reference herein.

In some embodiments, silicone oil is prepared by a ring-opening polymerization of strained cyclic silicones.

The kinematic viscosity of different types of base oils, such as polydimethylsiloxane base oils or a high-density silicone oils that comprise polydimethylsiloxane, can be expressed in centistokes (cSt). In one embodiment, the kinematic viscosity of the base oil ranges from about 100 to about 10,000 cSt, from about 200 to about 9,500 cSt, from about 300 to about 9,000 cSt, from about 400 to about 8,500 cSt, from about 500 to about 8,000 cSt, from about 600 to about 8,500 cSt, from about 700 to about 7,000 cSt, from about 800 to about 6,500 cSt, from about 900 to about 6,000 cSt, from about 950 to about 5,500 cSt, from about 1,000 to about 5,000 cSt, from about 1,100 to about 4,900 cSt, from about 1,200 to about 4,800 cSt, from about 1,300 to about 4,700 cSt, from about 1,400 to about 4,600 cSt, from about 1,500 to about 4,500 cSt, from about 1,700 to about 4,300 cSt, from about 1,900 to about 4,100 cSt, from about 2,000 to about 4,000 cSt, from about 2,200 to about 3,800 cSt, from about 2,400 to about 3,600 cSt, from about 2,600 to about 3,400 cSt, or from about 2,800 to about 3,200 cSt. In some embodiments, the kinematic viscosity of the silicone oil may be about 500 cSt, about 600 cSt, about 700 cSt, about 800 cSt, about 900 cSt, about 1,000 cSt, about 1,100 cSt, about 1,200 cSt, about 1,300 cSt, about 1,400 cSt, about 1,500 cSt, about 1,700 cSt, about 1,900 cSt, about 2,100 cSt, about 2,300 cSt, about 2,500 cSt, about 2,700 cSt, about 2,900 cSt, about 3,100 cSt, about 3,300 cSt, about 3,500 cSt, about 3,700 cSt, about 3,900 cSt, about 4,100 cSt, about 4,300 cSt, about 4,500 cSt, about 5,000 cSt, about 6,000 cSt, about 7,000 cSt, about 8,000 cSt, about 9,000 cSt, about 10,000 cSt.

References to kinematic viscosity as used herein refer to those measured using a kinematic viscometer. One representative kinematic viscometer is the Anton Parr Kinematic Viscometer SVM 1001 (Anton Paar GmbH), using the appropriate selection of speed settings as per the viscosity of the liquid.

In some embodiments, the oxygen-enriched tamponade composition can further comprise a drug. The drug can be a therapeutic, prophylactic, or both therapeutic and prophylactic drug. In some embodiments, the oxygen-enriched tamponade composition can be made by adding oxygen by the methods described herein to a silicone oil composition comprising a drug. In some embodiments, the silicone oil composition comprising a drug can be those described in U.S. Patent Application Publication No. US20,190,175497.

In some embodiments, the silicone oil can include or exclude a fluoropolymer. In some embodiments, the silicone oil can be those described in U.S. Pat. Nos. 5,336,487, or 6,703,378, each of which is herein incorporated by reference in their entirety.

### Increasing Oxygen Levels in Tamponades

The presence of any gas in a silicone oil tamponade for post-surgical repair was considered undesirable in the art because the gas can affect the volume of the silicone oil delivered to the eye and also introduce visual distortion. Many methods of using silicone oil as a post-repair tamponade involve a degassing step to remove gasses from the silicone oil tamponade, suggesting the presence of oxygen gas in a liquid tamponade is discouraged in the art.

Described herein are a variety of methods to increase the oxygen levels in the tamponades of this disclosure to oxygen levels higher than that of the tamponades at ambient conditions. While oxygen may be present in silicone oil or fluorocarbon at ambient conditions, the oxygen levels will be limited by several factors including temperature, solubility, humidity, and ambient air pressure. The oxygen levels in the silicone oil or fluorocarbon liquids at ambient conditions will also be affected by the equilibrium conditions. The inventors have developed methods of increasing the oxygen content in the silicone oil or fluorocarbon liquids as described herein such that the oxygen-comprising tamponades of this disclosure comprise oxygen levels higher than that of comparable silicone oils or fluorocarbon liquids at ambient conditions.

The dissolved oxygen in the silicone oil can be raised from 1% in conventional silicone oil to about 22-42% dissolved oxygen after oxygen enrichment. There is continued release of oxygen from the oxygen-enriched silicone oil for up to 7 days after initial oxygen infusion.

Silicone oil or perfluorocarbon compositions have a significantly greater solubility of oxygen than aqueous solution (approximately 20 times for silicone oil), which can result in the significant enhancement of oxygen availability to the retina during the post-operative period. Furthermore, increasing the partial gas pressure of the oxygen during mixing, creation of microbubbles or regular oxygen bubbles within the viscous silicone oil or perfluoron (fluorocarbon) and adjusting the temperature can further increase the storage and delivery of oxygen to the retina in the post-operative period.

Advantages of the oxygen-enriched tamponade compositions of the present invention include an economical, highly effective and convenient way to provide oxygen to healing retinal tissue after surgery.

In some embodiments, oxygen can be mixed into the silicone oil by the methods described herein. In some embodiments, oxygen can be solubilized, insolubilized, or both, within the tamponade composition. When the oxygen is solubilized, the level of oxygen dissolved in the silicone oil can be 20-2000% more compared to the oxygen levels in ambient (untreated) silicone oil. In some embodiments, the oxygen content in the tamponade composition can range from 0.2 mg O₂/L of tamponade composition up to 2247 mg O₂/L (Johnson et al., 2022 Meet. Abstr. MA2022-01, 1665; DOI 10.1149/MA2022-01381665mtgabs). In some embodiments, the oxygen concentration in the tamponade composition can range from about 1 mg O₂/L to about 1000 mg O₂/L, about 5 mg O₂/L to about 500 mg O2/L, 10 mg O₂/L to about 1000 mg O₂/L, 10 mg O₂/L to about 500 mg O₂/L, 20 mg O₂/L to about 1000 mg O₂/L, 20 mg O₂/L to about 500 mg O₂/L, 50 mg O₂/L to about 1000 mg O₂/L, 50 mg O₂/L to about 500 mg O₂/L, 100 mg O₂/L to about 1000 mg O₂/L, 100 mg O₂/L to about 500 mg O₂/L, 500 mg O₂/L to about 1000 mg O₂/L, or 1 mg O₂/L to about 5000 mg O₂/L, or between any of the aforementioned ranges.

In some embodiments, the concentration of the oxygen content in the oxygen-enriched tamponades of this disclosure can be about 0.1 mg O₂/L, 0.2 mg O₂/L, 0.3 mg O₂/L, 0.4 mg O₂/L, 0.5 mg O₂/L, 0.6 mg O₂/L, 0.7 mg O₂/L, 0.8 mg O₂/L, 0.9 mg O₂/L, 1 mg O₂/L, 2 mg O₂/L, 3 mg O₂/L, 4 mg O₂/L, 5 mg O₂/L, 6 mg O₂/L, 7 mg O₂/L, 8 mg O₂/L, 9 mg O₂/L, 10 mg O₂/L, 20 mg O₂/L, 30 mg O₂/L, 40 mg O₂/L, 50 mg O₂/L, 60 mg O₂/L, 70 mg O₂/L, 80 mg O₂/L, 90 mg O₂/L, 100 mg O₂/L, 200 mg O₂/L, 300 mg O₂/L, 400 mg O₂/L, 500 mg O₂/L, 600 mg O₂/L, 700 mg O₂/L, 800 mg O₂/L, 900 mg O₂/L, 1g O₂/L, 2g O₂/L, 3g O₂/L, 4g O₂/L, 5g O₂/L, 6g O_{2/}L, 7g O₂/L, 8g O₂/L, 9g O₂/L, 10g O₂/L, 20 g O₂/L, 30 g O₂/L, 40 g O₂/L, 50 g O₂/L, 60 g O₂/L, 70 g O₂/L, 80 g O₂/L, 90 g O₂/L, 100 g O₂/L, or a concentration between any of the aforementioned concentrations.

In some embodiments, oxygen in insolubilized or substantially insolubilized in the tamponade composition in the form of bubbles or microbubbles. The oxygen content of the silicone oil can be comprised of both dissolved oxygen in the silicone oil and in microbubbles within the silicone oil.

In some embodiments, the silicone oils of this disclosure can be degassed prior to use to increase the oxygen content for the total gas solubility in the oil. Degassing methods can include or exclude: centrifugation, freeze-pump-thaw, or passing the solution through an appropriate membrane. The degassed viscous tamponade compositions can then be oxygen-enriched with pure oxygen gas by a variety of methods as described herein.

Oxygen enrichment methods can include or exclude: static inline mixer or a rotor-based mixer with moving parts with stirring mix oxygen gas into the silicone oil. These methods, may, however, result in large oxygen bubbles. In some embodiments, methods of enriching the oxygen content in the tamponades of this disclosure can include introducing oxygen gas as microbubbles. Microbubbles would be a more desirable technique wherein by gradually injecting oxygen gas with a micro-canula within a larger cannula filled with silicone oil and simultaneously pushing the silicone oil and the gas in the same direction, microbubbles of oxygen gas in the order of <50 microns can be introduced into the silicone oil. In some embodiments, the microbubble-containing silicone oils can be made by the methods referred to in U.S. Patent No. 9,802,165, incorporated by reference herein.

In some embodiments, oxygen enrichment of the tamponades of this disclosure can include applying ultrasound to an oxygen-rich environment with slow infusion of oxygen gas into the silicone oil to create smaller-sized oxygen bubbles by means of cavitation. Without being bound by theory, increasing the partial gas pressure of the oxygen can increase the storage/solubility of oxygen gas in the silicone oil because the solubility of oxygen gas in silicone oil obeys Henry's law.

In some embodiments, methods of enhancing the oxygen content in the tamponade compositions of this disclosure can include lowering the viscosity of the silicone oil, and raising the temperature of the silicone oil during mixing and storage.

Conventional methods in rheology related to mixing of gas into heavy fluids include sparging or directly bubbling the gas into the liquid. In one embodiment, a static inline mixer can be used to mix oxygen gas into the silicone oil to increase the oxygen content of said oil above that of ambient conditions. This method enhances oxygen content by creating intertwining flow pathways which increase surface area for mixing of gas and fluid. Dynamic mixers with rotors or other moving parts are used in commercially available mixer where stirring and agitation can increase oxygen solubility into heavy fluids like silicone oil. Bubble sizes tend to be larger using this method of mixing.

Modern techniques of gas-fluid mixing involve creation of microbubbles. These bubbles are usually sub-millimeter in size (<50 um) and can be created with a variety of techniques. Microbubbles are unlikely to cause any significant visual disturbances because of their small size-small cholesterol crystals in the vitreous present in asteroid hyalsosis generally do not cause visual disturbances. A representative method involving oxygen microbubble formation in tamponades involves passing oxygen gas at a slow rate in a microcanula which rests within a large canula filled with silicone oil. By simultaneous passing both fluid and gas at a slow rate in the same direction, microbubbles can be introduced into the heavy liquid like silicone oil. Representative methods of making microbubbles are described in Churchman et al. (Microsystems & Nanoengineering volume 4, Article number: 17087 (2018)).

The size of microbubbles will depend on the mixing method deployed, the sizes of the bubbles created in the mixture may vary, from microbubbles (<50 um) to regular-sized bubbles and to large bubbles (>5 mm) usually based on the degree of agitation involved in the mixing process.

Ultrasonic cavitation can also be employed to create the agitation necessary to introduce small-sized oxygen bubbles into silicone oil. Ultrasound can be used to create cavitation within the silicone oil. By simultaneously infusing oxygen gas into the silicone oil, the cavitation generated by ultrasound can trap oxygen within the silicone oil and generate the desired oxygen enrichment. This cavitation technique can be used in combination of rotor-based mixers.

Performing mixing in an environment with high oxygen gas content and increasing the partial gas pressure oxygen gas can enhance the solubility of the oxygen gas into silicone oil in the mixing method previously described.

Biocompatible surfactants may be introduced to enhance the stability and efficiency of bubble formation and further increase oxygen solubility within silicone oil. Some examples of surfactants include sodium stearate, Pico-Surf, Tween-80, glycolipids, etc. However, due to possible effects on emulsification of silicone oil with addition of surfactant, this approach may not be employed in the ideal product example.

Solubility of oxygen is approximately 20× (twenty-fold) greater in silicone oil than in aqueous of the eye. Conventional mixing, microbubbles and/or ultrasonic cavitation can further increase the oxygen solubility in silicone oil and create a continuous-release oxygen-rich environment for the healing retina after surgery.

As a representative example, using 1000 Cs silicone oil or 5000 Cs silicone oil currently available on the market, oxygen infusion and mixing with silicone oil can be achieved in a variety of static and dynamic mixing, sparging, microbubble creation and ultrasound cavitation techniques. The mixing process and bubble creation process can be enhanced by raising the temperature, generally around 55 degree Celsius. Oxygen is infused into the silicone oil at an increased gas pressure chamber which is generally at around 100-2000 psi.

Similar methods to those for enriching the oxygen content of silicone oils can be applied to create an oxygen-enriched perfluorocarbon tamponade.

The process to produce oxygen-enriched tamponade compositions of the present invention is described in still greater detail in the examples that follow.

### EXAMPLES

### Example 1

### Direct Oxygen Gas Infusion or Sparging

10 cc of silicone oil of 5000 Cs viscosity is put in in enclosed vial with small gas inlet and outlet. Medical-grade filtered oxygen gas is infused or sprayed into the bottom of the enclosing through a gas inlet for 20 minutes. There is noted to formation of small bubbles within the silicone oil. Using a standard dissolved oxygen meter, the dissolved oxygen(DO) content of the silicone oil is measured to be about 22% of dissolved oxygen compared to 1% dissolved oxygen in silicone oil without oxygen enrichment. This is performed under atmospheric pressure but one would expect a higher degree of oxygen solubility at increased oxygen gas pressure or higher temperature. The oxygen-enriched silicone oil provides a significant release of oxygen over the initial 48 hour period but the release is continued for up to 7 days where the enriched silicone oil maintained a DO of >2% 7 days after the initial oxygen infusion.

### Example 2

Mixing with Commercial Rotor Mixer

Silicone oil of 5000 Cs is processed with an inline commercial rotor-mixer. The silicone oil is continuously stirred while medical grade oxygen gas is infused into the enclosed chamber at increased pressure (in the range 100-3000 psi) for 30 minutes. Using a standard dissolved oxygen meter, the dissolved oxygen(DO) content of the silicone oil is measured to be about 42% of dissolved oxygen compared to 1% dissolved oxygen in silicone oil without oxygen enrichment.

### Example 3

### Microbubble Generator

Silicone oil of 5000 Cs is processed with a commercial microbubble generator. The generator directly injects microbubbles of medical grade oxygen gas into the silicone oil in an enclosed chamber for 30-60 minutes at an elevated gas influsion pressure.

### Example 4

Microbubble Creation with Micro-Canula

Medical grade oxygen gas is slowly infused in a micro cannula. The microcannula is embedded within a larger cannula where silicone oil of 5000 Cs viscosity is infused gradually. Thus there are two lumens. The inner lumen contains oxygen gas and the outer lumen contains silicone oil and both silicone in and oxygen gas travels on the same direction. Mixing occurs and microbubbles are created as the inner lumen ends and oxygen gas microbubbles exits and merges with the surrounding silicone oil in the larger lumen. Microbubbles of oxygen gas are created within the silicone oil.

### Example 5

### Bubble Formation by Ultrasonic Cavitation

Medical grade oxygen gas is infused in an enclosed bath which is agitated with ultrasound. Cavitation is created by ultrasonication and oxygen gas is incorporated by the small bubbles within the 5000 Cs silicone oil.

### Example 6

Treatment of a subject with a retinal hypoxia.

A subject presenting a retinal hypoxia condition is administered an oxygen-enriched tamponande composition of this disclosure. An incision into the posterior chamber of an eye of a subject is first made, then the oxygen-enriched tamponade is presented to the eye at the site of the incision. The retina condition is measured by fundus photography at selected time points throughout the treatment, including at baseline. After a selected period of time after administration (e.g., 7 days), the condition of the retina as monitored by fundus photography is expected to show recovery of the retina or an improvement of the retina.

What is claimed is:

1. An oxygen-enriched tamponade composition comprising oxygen at a concentration higher than that of ambient concentration and a base oil selected from a silicone oil, a fluorocarbon, or mixtures thereof.

2. The oxygen-enriched tamponade composition of claim 1, wherein the oxygen has been exogenously added to said base oil.

3. The oxygen-enriched tamponade composition of claim 1, wherein the percentage of dissolved oxygen after oxygen enrichment ranges from at least 20-52% compared to 0-1% for non-oxygen enriched base oil.

4. The oxygen-enriched tamponade composition of claim 1, wherein the oxygen was added to the base oil at a temperature less than 0 degrees Celsius.

5. A method of preparing an oxygen-enriched tamponade composition comprising adding exogenous oxygen to a base oil selected from a silicone oil or a fluorocarbon at a temperature of less than 0 degrees Celsius.

6. The oxygen-enriched tamponade composition of claim 2, wherein the exogenous addition of oxygen is performed by directly infusing medical grade oxygen gas into the fluid tamponade.

7. The oxygen-enriched tamponade of claim 6, wherein the directly infusing medical grade oxygen gas into the fluid tamponade is performed by sparging said gas into the base oil.

8. The oxygen-enriched tamponade composition of claim 2, wherein the exogenous addition of oxygen is performed by inline static mixing of oxygen gas into the base oil.

9. The oxygen-enriched tamponade composition of claim 2, wherein the exogenous addition of oxygen is performed by dynamic mixing injected oxygen gas with a rotor to stir the base oil and injecting oxygen gas simultaneously.

10. The oxygen-enriched tamponade composition of claim 2, wherein the exogenous addition of oxygen is performed by injection of microbubbles comprising oxygen into the base oil.

11. The oxygen-enriched tamponade composition of claim 10, wherein the size of the microbubbles is less than 50 microns.

12. The oxygen-enriched tamponade composition of claim 10, wherein the microbubbles are formed by injecting oxygen gas into a micro-cannula configured to be embedded within a surrounding cannula which comprises the base oil followed by expelling the mixture from the cannulas to yield the oxygen-enriched tamponade composition.

13. The oxygen-enriched tamponade composition of claim 12, wherein the micro-cannula and surrounding cannula each comprise a separate lumen, and the lumen are configured to merge at the expelling point.

14. The oxygen-enriched tamponade composition of claim 13, whereby mixing of the microbubbles occurs when the micro-cannula and surrounding cannula lumen merge together.

15. The oxygen-enriched tamponade composition of claim 2, wherein the exogenous addition of oxygen is performed by the creation of cavitation or bubbles by ultrasonicating the base oil while simultaneously injecting oxygen gas into said base oil.

16. The oxygen-enriched tamponade composition of claim 2, wherein the exogenous oxygen pressure is added at a range from 20-3000 psi.

17. The oxygen-enriched tamponade composition of claim 2, wherein the exogenous oxygen pressure is added at a temperature ranging from 20-100 degrees Celsius.

18. A method of treating a retinal injury in a subject comprising the steps of:
a. creating an incision into the posterior chamber of an eye of a subject having a retinal injury; and
b. administering to said posterior chamber of the eye an oxygen-enriched tamponade of claim 1.

19. The method of claim 18, wherein the oxygen-enriched fluid tamponade is stored in air-tight vessel to maintain the oxygen content prior to its use as an ocular tamponade.

20. The method of claim 18, wherein the oxygen in the oxygen-enriched tamponade is continuously released for up to 7 days after administration.

21. The method of claim 18, wherein the oxygen in the oxygen-enriched tamponade is continuously released for up to 2 days after administration.

22. The method of claim 21, wherein 80% of the oxygen in the oxygen-enriched tamponade is continuously released for up to 2 days after administration.
